(19) United States
(12) Patent Application Publication
(10) Pub. No.: US 2016/0008568 A1
(43) Pub. Date: Jan. 14, 2016

(54) RELAXATION APPARATUS AND METHOD

(71) Applicants: Sarah Beth Attia, Santa Monica, CA (US); Matthew Markert, Kansas City, MO (US)

(72) Inventors: Sarah Beth Attia, Santa Monica, CA (US); Matthew Markert, Kansas City, MO (US)

(21) Appl. No.: 14/499,069

(22) Filed: Sep. 26, 2014

Related U.S. Application Data
(63) Continuation-in-part of application No. 14/183,596, filed on Feb. 19, 2014.
(60) Provisional application No. 61/883,037, filed on Sep. 26, 2013.
(60) Provisional application No. 61/850,579, filed on Feb. 20, 2013.

(51) Int. Cl. A61M 21/00; A61B 5/00; A61M 16/00; A61M 21/02

(52) U.S. Cl. CPC A61M 21/0094; A61M 21/02; A61B 5/486; A61M 16/00; A61B 5/4854; A61M 2021/0027; A61M 2021/0044; A61M 2205/3303; A61M 2230/04; A61M 2230/42; A61M 2230/30; A61M 2230/08; A61M 2205/52

(57) ABSTRACT

A relaxation dome apparatus including a curved shell element which creates a microenvironment substantially surrounding the head, neck, and torso of a user person, wherein the interior surface of the shell facing the user person forms a relatively smooth regular curved surface. The apparatus includes an individual seating space, audio transducers configured to produce at least two sound signals proximate the user person's head, and a user interface to guide the user in obtaining a personal relaxation experience without need for an attendant, and for the purposes of collecting data. The apparatus may include combinations of visible light stimuli, biofeedback or neurofeedback data collection sensors, audio tracks which may include monaural, isochromic or binaural beats, and microcrystalline ceramic tiles. A database of aggregated user data and an Internet connection may each enable broadened therapy experiences for users.

### CROSS-REFERENCE TO RELATED APPLICATIONS

This application claims the benefit of Provisional Application No. 61/883,037, entitled “RELAXATION APPARATUS AND METHOD” filed on 26 Sep. 2013, and is a continuation-in-part application of U.S. patent application Ser. No. 14/183,596, entitled “RELAXATION DOME APPARATUS” filed on 19 Feb. 2014 which claimed the benefit of Provisional Application No. 61/850,579 filed on 20 Feb. 2013. All are incorporated herein by reference.

### BACKGROUND

Conventional environments used to offer a space conducive for relaxation, such as yoga and spa or retreat centers, or meditation classes, are typically located in permanent immovable environments that require the participant to travel to an actual establishment or separate destination.

### SUMMARY

A method includes assisting an individual to achieve a relaxed mental state whereby the individual is presented a session comprising a predetermined combination of simultaneous auditory and visual stimuli while being inside a self-contained private space. The session may include an uninterrupted duration of at least twelve minutes. There may be a visual stimulus of a substantially deep-blue color and the auditory stimulus includes binaural beats approximating the frequencies of beta brain waves and gamma brain waves and theta brainwaves. The visual stimulus may be a substantially light-blue color and the auditory stimulus includes binaural beats approximating the frequencies of beta brain waves and gamma brain waves and theta brain waves. The visual stimulus may be a substantially green color and the auditory stimulus includes binaural beats approximating the frequencies of delta brain waves. The visual stimulus may be a substantially green color and the auditory stimulus includes binaural beats approximating the frequencies of theta brain waves. The visual stimulus may be a substantially light-blue color and the auditory stimulus includes binaural beats approximating the frequencies of alpha brain waves. The visual stimulus may be a substantially fuchsia color and the auditory stimulus includes binaural beats approximating the frequencies of theta brain waves. The visual stimulus may be a substantially deep-blue color and the auditory stimulus includes binaural beats approximating the frequencies of theta brainwaves.

A stand-alone relaxation unit may include an binaural beat audio generator, a light therapy device, a chair, and a housing integrated with the chair, the housing defining a partially-enclosed personal space. The unit may include a controller adapted to select an audio content based on a biofeedback of a user, the controller further adapted to cause the audio generator to emit the audio content. The controller may be further adapted to select a light therapy content based on a biofeedback of the user, and to cause the light therapy device to emit the audio content. The light therapy device may include an LED coupled with a dome of the housing. The unit may include a ventilator. The biofeedback may be a datum selected from the group consisting of a pulse datum, a rate of respiration datum, a blood pressure datum, and a neurosensory datum. The controller may be adapted to cause the biofeedback to be recorded. The unit may include a TRH4 disc coupled with the dome. The controller may be adapted to select an audio content based on a recorded biofeedback of the user of a prior session.

### BRIEF DESCRIPTION OF DRAWINGS

FIG. 1 is a front (side elevation) view of a portable relaxation dome.

FIG. 2 is a top interior (plan) view of the relaxation dome shown in FIG. 1.

FIG. 3 is a rear (end elevation) view of the portable relaxation dome embodiment shown in FIG. 1.

FIG. 4 is a perspective view of a dome lid hinged to the back of a chair as an alternative to the completely enveloping enclosure of FIG. 1.

FIG. 5 shows the hinge of a dome lid as located at back of a chair—closed position.

FIG. 6 shows dome lid in open position.

FIG. 7 shows active visible light emitters around dome lid periphery.

FIG. 8 is a flow chart illustrating user identification with menus adjusted to provide a personalized user experience.

FIG. 9 is a flow chart illustrating the session selection process upon identification of user status.

FIG. 10 is a flow chart illustrating a process proactively assisting the user in selection of an audio content track.

FIG. 11 is a color gambit chart for mapping desired color perception to visible light emitter outputs.

FIG. 12 is a flow chart illustrating dynamic modification of control device outputs to the user microenvironment based upon real-time physiological biofeedback data combined with historical data of both the specific user and general populations.

### DESCRIPTION

Disclosed herein is a portable relaxation dome or apparatus that integrates audio stimuli (relaxation program content), optional visual stimuli, and biofeedback technology for creating personal psychological relaxation sessions. The apparatus may communicate with a server. The server may host a database for each user, and may synchronize an individual user's biofeedback data, thereby enabling the user to track their progress during, after, and between sessions, and provide them an ability to share their data with a community of users.

Biofeedback or neurofeedback information may be obtained from suitable sensors applied by the user at appropriate locations upon their body so as to provide physiological or neurophysiological data. This obtained data may be used to modify various aspects of the user experience presented by the relaxation apparatus. At other points in this document to reduce redundancy or for brevity or space (as is the case for included infographics), biofeedback may be used as a definition which includes and assumes neurofeedback.

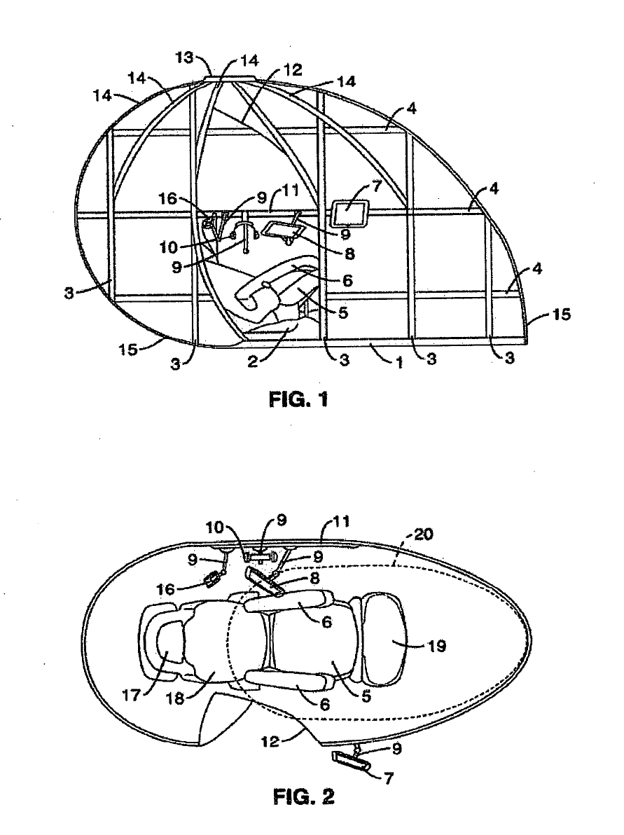

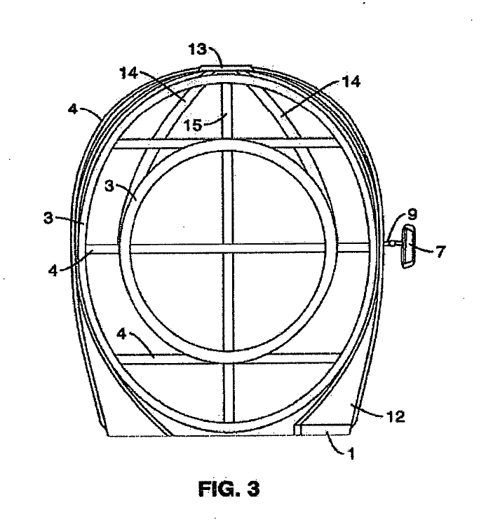

A representative example of a relaxation dome apparatus is shown in FIG. 1, FIG. 2, & FIG. 3. Those Figures show a base/baseboard 1, reclining chair base 2, vertical trim pieces 3, horizontal trim pieces 4, a reclining chair with a seat 5, adjustable arm rests 6 for the reclining chair, an outside (mounted) interface/computer/monitor 7, an inside interface/computer/monitor 8, an articulating arm 9 for a monitor and headphones, wireless headphones 10, a connecting bar 11 for the articulating arm, a door flap region 12 where a zipper, hook and loop fastener, or similar closure technique may be used (the door is shown removed—open), a top connection hub 13 for frame pieces/trim pieces, vertical trim pieces 14 connecting to the top connection hub, a biofeedback device 16 (including earlobe clip and wire) and possible optional LED eyeglasses, a relaxation chair head rest 17, a relaxation chair back rest 18, a relaxation chair foot rest 19, and a base perimeter 20 (to indicate where the base of reduced extent appears in the top plan view FIG. 2).

The semi-enclosed unit may include a dome-shaped space inside which a user can sit comfortably in a semi-reclined position, and listen to a guided audio content track that may or may not include the use of binaural beat brainwave technology, which may instruct the user, or entrain the user, concerning methods to relax based upon their chosen purpose (goal): mental focus, positivity and abundance, peace of mind, weight loss, stress reduction, quitting smoking, letting go of fear related to anxieties, attracting or manifesting desired outcomes in one's life, and a broad variety of other tracks that are designed to permit the user to identify a chosen target of achievement for particularly desirable mental outcomes. Light therapy display glasses may or may not be integrated into the apparatus. Controlled sources of visible colored light may illuminate the interior surface of the domed enclosure in a determined manner based upon the guided audio content, or according to other information provided by or from the user including personal selection, or biofeedback information.

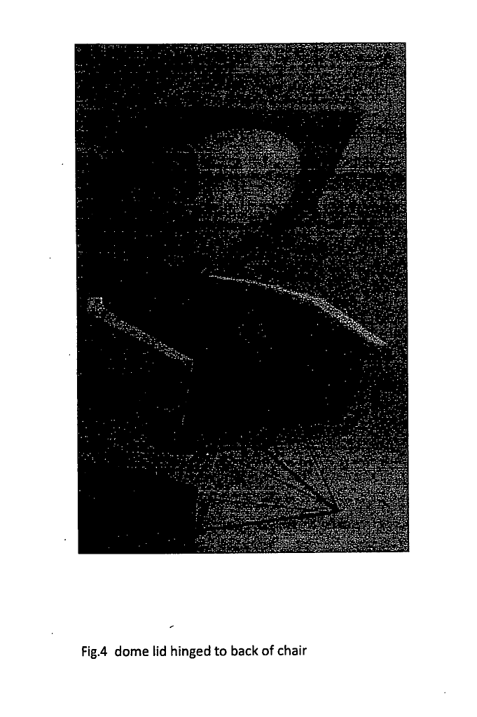

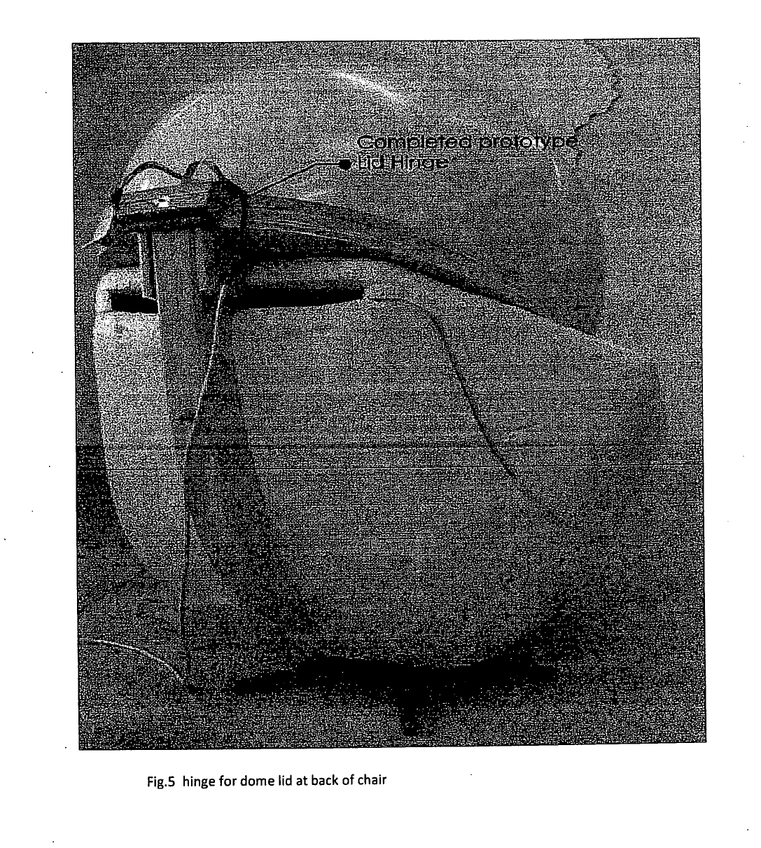

The relaxation dome apparatus is intended to create a microenvironment that people can enter, at their leisure in offices and retail or service environments, to decompress from their lives and realign their mind with their body in a session chosen to match a desired outcome for 20 or 30 minutes. The design of the portable relaxation dome is that of a collapsible, portable structure which has been designed for instructional sessions of guided relaxation integrated with biofeedback, at any time, and in virtually any environment, private or commercial. As can be seen in FIGS. 1, 2, &3, the dome structure may be sculpted by vertical trim pieces 14 is held in a vertical position resting directly on the floor, or elevated above the chair 5, while the microenvironment might alternatively be created by a dome lid hinged to the back of the chair 5 and scalloping over the top of the chair 5 as further explained with respect to FIG. 4. The portable relaxation dome structure includes an enclosure having a bottom wall, a front wall, a pair of opposing sidewalls, and an opening sidewall feature, or door flap, enabling a closable opening for user ingress and egress.

The main enclosure material used to construct the exterior structure of the relaxation dome may be manufactured using a polyurethane molded plastic, or other moldable material or via any other conventional technique. The dome can therefore also be made out of a variety of synthetic, hard-shell materials. The portable relaxation dome is able to collapse by use of interior mounted hinges constructed out of aluminum tubing, but can also be built out of other moldable materials including bendable plastic tubing. The construction details of the invention as shown in FIGS. 1-3 are that the tubing includes wider aluminum paneling scalloping the dome from the front to the back, in the vertical direction, about ten (10″) inches in width. In further detail, the same width aluminum paneling may frame the dome horizontally, from side to side as well. There may be narrower aluminum paneling of similar aesthetic, only closer to four to six (4″-6″) inches in width, running diagonally from back to front and side to side, in between the larger horizontal and vertical pieces. The bottom 1 of the portable relaxation dome may carry four casters, or wheels, or sliders which allow the portable relaxation dome to be taken apart, moved, and reassembled when needed.

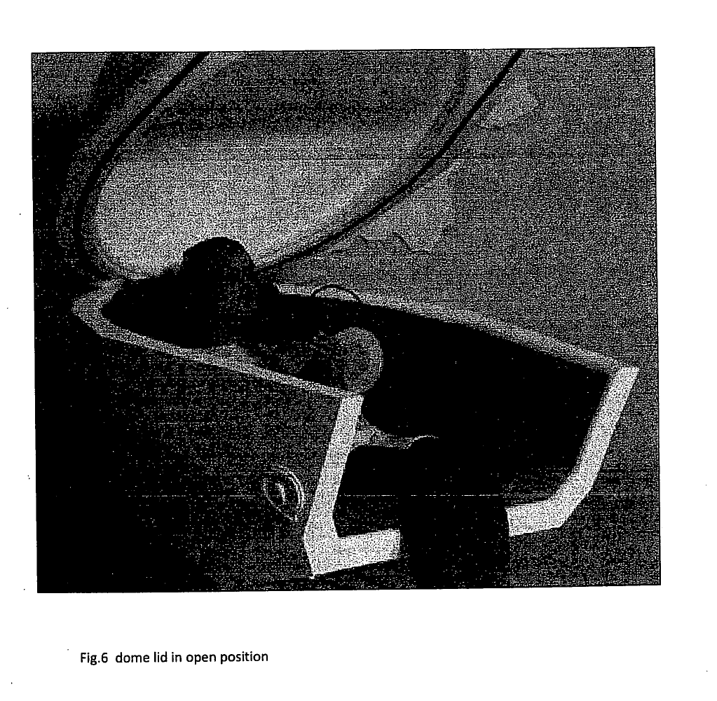

In other embodiments the dome enclosure may comprise, in lieu of a large dome structure inside which the user sits fully enclosed, an egg-shaped, ovoid, elliptical, or hemispherical dome lid that clamps at the back of the chair and lifts and lowers to allow the user's ingress and egress. In another representative example, illustrated in FIGS. 4&5 the dome lid itself is roughly three and one-half feet long, and both wide and long enough to cover the individual's torso in order to allow comfortable seating, such as about two to four feet wide, situated four to five feet high, and with rounded edges, although the shape might contour to an egg-like form, with either a horizontally or vertically stretched oval structure, or may instead be more spherical in design, narrower or wider than the example. As seen in FIG. 6 the dome lid covers the user's torso, head and neck regions, instead of enclosing their entire body.

The exterior of the dome structure can be constructed out of any soft fabric such as hemp, or burlap, or a synthetic material stretched tautly over an internal frame structure that creates the shape of a more or less oval or egg shape. Otherwise, the exterior of the dome can be constructed out of a hard shell using a synthetic moldable material, as in the case of the dome lid. On the perimeter outside and perimeter inside of the dome (either or both), may be two different sizes of aluminum paneling that form the shape of the dome itself, for the visual contrast aesthetic of aluminum being placed against the hemp material. Regarding ingress and egress to the dome environment, a sidewall opening may be provided in the case of an all-enveloping dome structure as FIG. 1, or a hinged dome lid may clamp at the back of the chair, as in FIG. 5, in order to move up and down, enabling the user to enter and exit the dome environment. Therefore, alternatively in further detail, the dome environment may be realized as a smaller dome lid placed above the head and torso of the user, and connected to the reclining chair. In order to enter and exit the dome, the user would open the dome lid upward in order to sit down and duck their head underneath the dome lid and then pull the dome lid down over their head, neck and torso.

The apparatus includes a seating device (e.g. element 2 in FIG. 1), chair or seat or lounge, held in a substantially level position elevated above the floor, typically by four upright legs attached to the base of the chair. The seating device is of a type that enables the user to recline, and also relieves pressure from the user's spine, so as to be comfortably recumbent although generally not supine. The interior of the portable relaxation dome may include a reclining style chair, noise cancellation stereo headphones, possibly a shelving unit which may contain a spray containing waters that have been tuned to chi-based healing frequencies, and microcrystalline ceramic tiles (such as BioSyntonie available from Terre de Lys located at 11 Route du Coq Gaulois-Digeon 80290, Morvillers-Saint-Saturnin, France) placed in the base and interior of the relaxation dome apparatus. The relaxation apparatus may be positioned with its front facing ‘true north’ as determined through use of a compass at the time the apparatus is installed in its location. A compliment of microcrystalline tiles may be arranged in a symmetrical pattern surrounding a single central tile. For example, a compliment of five type TRH4 disc-shaped tiles would be placed equidistant from each other under a seat cushion of the chair, four being near the perimeter of a square-shaped piece of cardboard, with the ‘fleur de lys’ symbol pointing upward, and positioned under the seat cushion with a piece of fabric or other suitable material encasing them to protect from casual exposure regarding their location or placement. Alternatively, nine tiles would be positioned with individual items located in the directions of north, south, east, and west having a flower facing up; while tiles running on the diagonals would have a picture facing up; and a tile in the middle would have a flower facing up.

Users receptive to the Eastern-based principles found in the concept of ‘energy medicine’ may appreciate that there has been careful placement of tiles, and similar items, within the periphery of the relaxation apparatus in locations intended to maximize impact on the users' experience. The apparatus user interface (or suitable signage) may inform users that the device has structurally integrated proprietary microcrystalline ceramic tiles, and inform them that the purpose of these tiles is to reduce or shield from any effects, or perceived effects, of electromagnetic waves or EMF emissions those tiles interact with. Actual or perceived electromagnetic field information from sources such as cell phones or nearby cell-phone towers, computers, microwaves, computer wireless router devices, or other technological media is a source of distraction for some users of relaxation experiences, and thereby the presence of microcrystalline tiles which may prevent penetration of said electromagnetic fields may improve the effectiveness of the technologies at work in the domed enclosure to achieve an optimum result for those users. Similar tiles are used by acupuncturists and placed on or around the body at certain meridian points to enable the flow of “chi” which is known in some Eastern medicine disciplines as the body's vital life force. Through the placement of these tiles in a specific configuration under the seat portion of the relaxation apparatus, and as potentially related to the position of the relaxation apparatus itself, the tiles mitigate excess electromagnetic radiofrequencies emitted from manmade devices, including those used to access internet waypoints. In order to avoid radio signals inside the apparatus, an external computing device may be mounted to the outside of the relaxation apparatus, which may communicate data collected from the biofeedback component inside the relaxation apparatus via a cable, while content presented to the user may be sourced for session selection via a cloud-based server.

The microenvironment created by the relaxation dome apparatus may buffer the user's senses from the chaotic and distracting sights and sounds typically found in the immediately adjacent world in office, retail, classroom, and similar public spaces. To effect audio isolation the dome may include sound adsorbing insulation in lieu, or in addition, to the mentioned electronic active noise cancelling headphones. Other components, e.g. a biofeedback device to measure the user's pulse or blood pressure, could also be plugged into an appropriate signal connecting outlet with ease, and then placed into an optional bracket (components 7 & 9) mounted on the outside or inside of the apparatus. More discussion of biofeedback information appears further below in this disclosure.

Audio content may or may not be influenced by information from biofeedback sensors within the apparatus. These sensors typically may be applied and worn by the user to measure pulse rate, respiration rate, blood pressure, heart rate variability, homoencephalography, skin temperature, galvanic skin response, or similar signals of the physiological or other user state or approximated state. This feedback data may be gathered and interpreted by a control device to modify the user experience within the session. The data may also provide the user with their individual information over time, or for each session, thereby enabling each user to track their progress and receive guidance and custom tailored sessions. The apparatus delivers the relaxation audio content, as well as integrates with the biofeedback sensors, and may communicate information through an integrated software application using a visual feedback device such as a tablet or LCD panel, through a web-based application, or through interface with a mobile operating system or smartphone. The information may be sent directly to, and the content received from, a suitable control device; moreover, the control device may be local to the apparatus or remotely located and accessible over the Internet using wired or wireless, or web-based connection methods as known in the computing art.

The chair components (seat 5, armrests 6, headrest 17, and leg-rest 19) may have frame members made of wood, or metal, or any other sufficiently rigid, strong, light weight material such as high-strength plastic, organic compost-based material, aluminum, or the like. Further, the various components of the chair (5, 6, 17, & 19) can be individually made of different materials while typically having a consistently similar aesthetic appearance imparted by upholstery covering and cushion padding. The structural aspects may render the device portable and exceedingly easy to assemble, maintain, and transport. It is easy to move the apparatus through the standard doorway of most houses or offices. The dome exterior and chair are designed to be relatively small and lightweight, neither piece weighing alone more than approximately 50 pounds. Moving such apparatus items typically requires a single person and typically at most two people when taking the device up or down stairs. Further, the apparatus items generally will pass through most doorways without any widening.

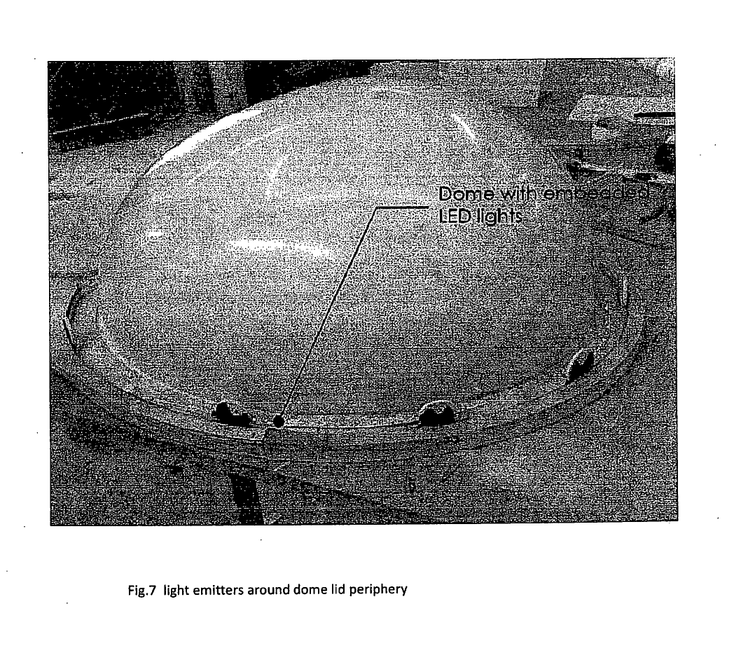

The interior surface of the enveloping dome structure, or alternatively the dome lid, is fashioned to be a relatively smooth regular curved surface free of obvious lines that might focus the visual attention of the user. The intention is to make it difficult for the user to focus their eyes on any particular aspect of the surface or a false horizon, thus obvious parting lines or alternative manners of opening are to be eschewed. The interior surface visible to the user is chosen to diffusely reflect light so as to surround the user and effecting a sort of loss of depth perception. Light therapy (or chromotherapy) may be provided by a plurality of active emitters of visible wavelength light typically located along the internal or external perimeter of the dome lid opening (see FIG. 7), or at approximately the equator of the user's peripheral field of view within the enveloping dome. The dispersed active emitters may be, for example, light emitting diodes (LEDs) of selected colors, or miniature incandescent lamps with selected filtering glass envelopes, or electroluminescent devices among other possibilities. The chosen colors of the selected active emitters are picked with consideration of the photometric response of the human eye to facilitate creating a controllably variable sensation of the dome environment being filled with distinct colors of light, each with varying intended correlating beneficial effects to enhance the properties of the chosen audio session.

A system is disclosed for guiding a user in obtaining a personal relaxation experience without need for an attendant clinician, comprising a relaxation dome apparatus including a curved shell element which creates a microenvironment substantially surrounding at least the head, neck, and torso of the user, wherein the interior surface of the shell facing the user forms a relatively smooth regular curved surface, and further including an individual seating space, audio transducers configured to produce at least two sound signals proximate to the user's head, and a user interface means in communication with a control means.

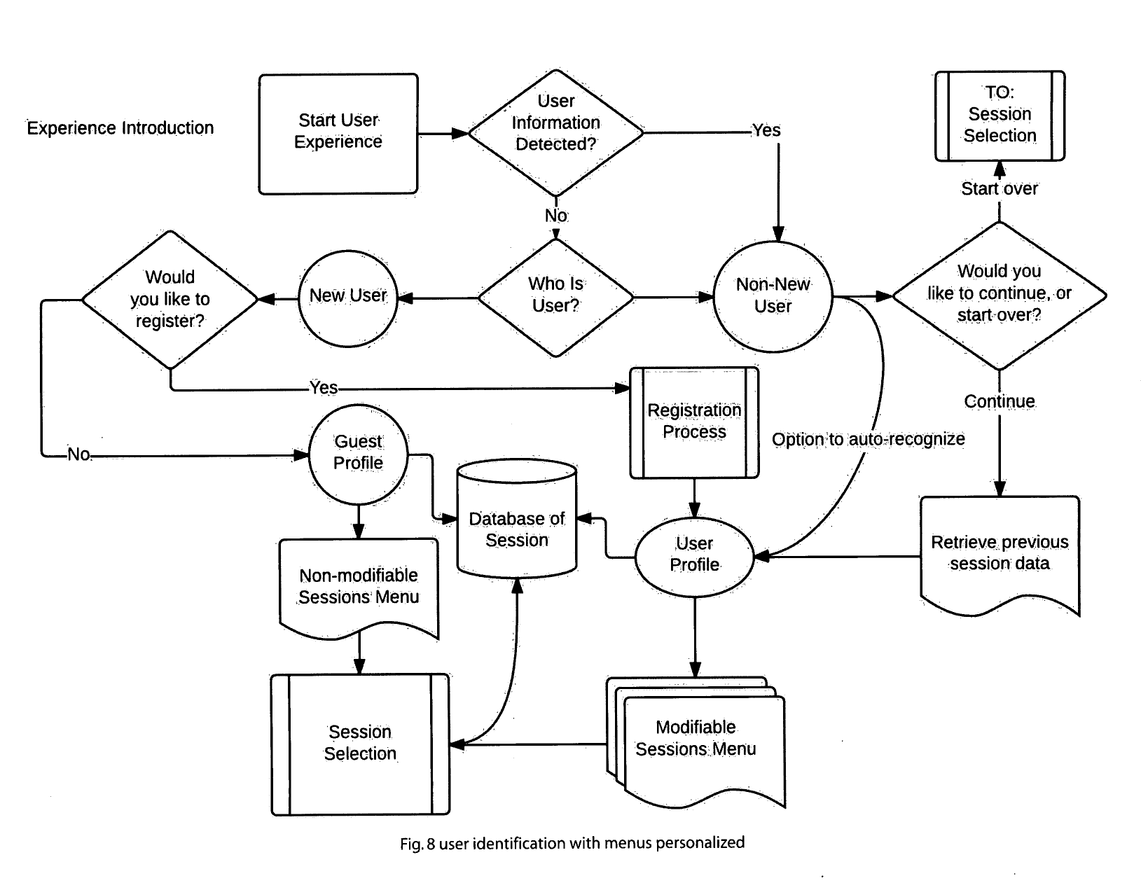

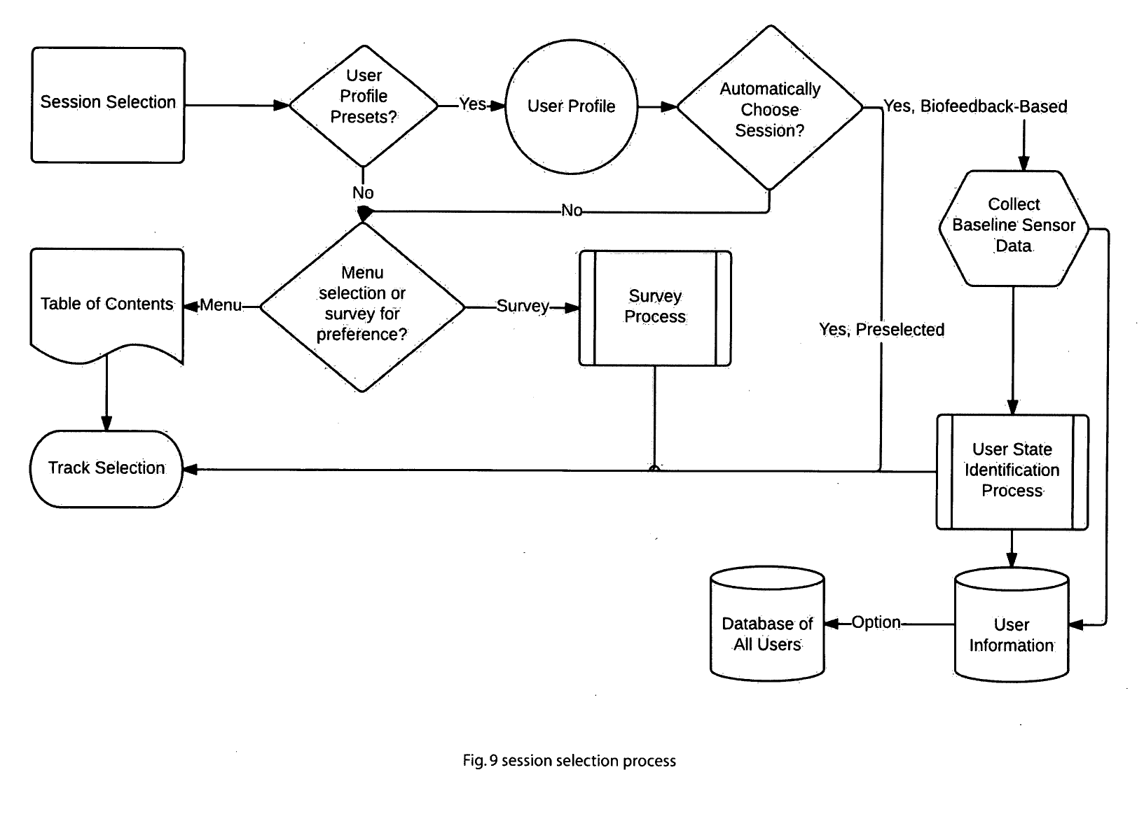

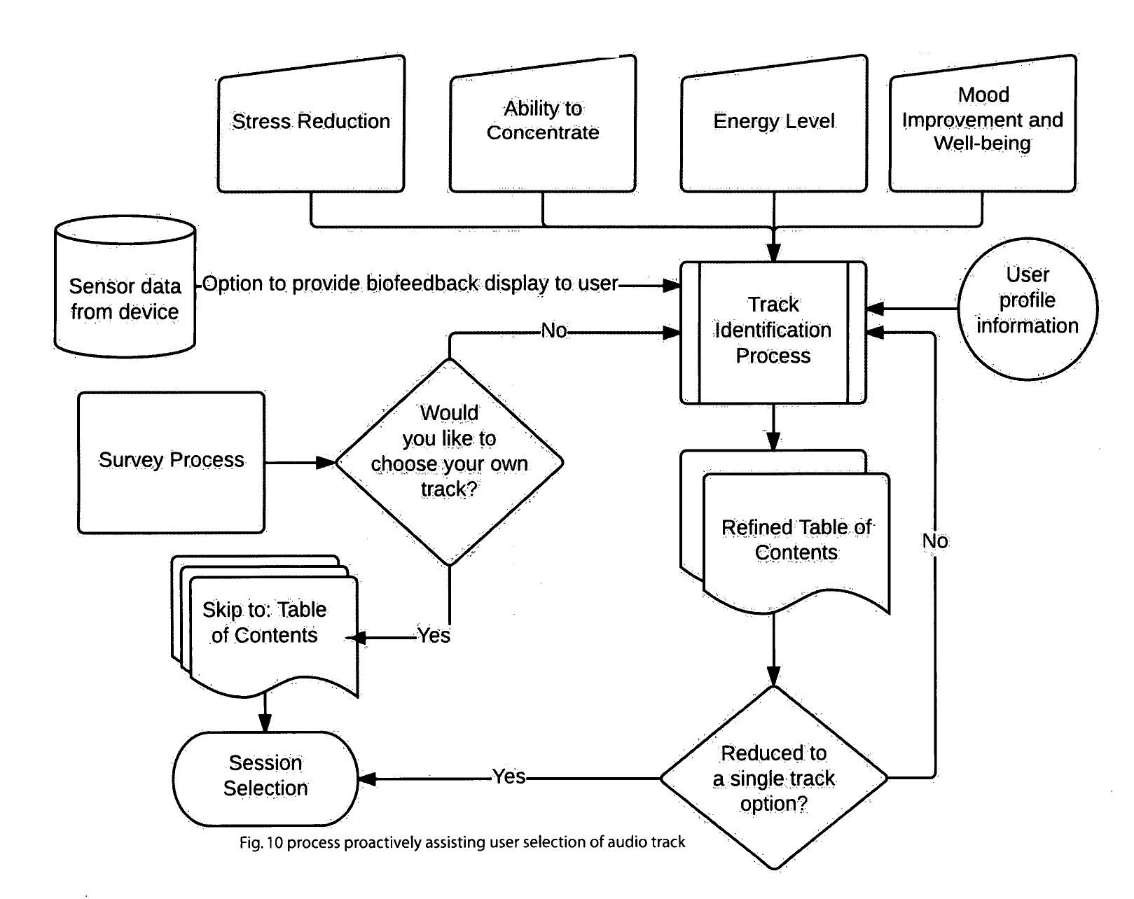

Upon entering the dome environment, as provided by a dome structure or a dome lid, and starting a relaxation session, a user is presented with a series of an initialization of questions through the action of an automated user interface. There is no monitoring person, or attendant required, nor desired since the apparatus enables self-directed relaxation sessions, and has a payment and scheduling capacity. An enhanced sense of personalized user experience is accomplished without employing a human attendant by automatically adjusting details of the initialization questions and subsequent menus based upon general and specific information known about the particular user. Details of the user experience may be refined according to the apparatus geographic location, user history, and potentially other considerations, as illustrated in the flow chart diagrams of FIG. 8, FIG. 9, and FIG. 10. In FIG. 8 the basic user experience begins with identification of user information, if that information may be known through previous exposure to the apparatus or through exposure to Internet World-Wide-Web or mobile operating system-based extensions of the apparatus. For new users, or for existing users who prefer to continue without identification via previous registries, registration may be elected or the user may continue with stand-alone sessions. In FIG. 9 the user is asked, through the user interface, whether the apparatus should choose a session type for the user (based upon known history and immediate biofeedback sensor data as available), or the user wishes to complete a survey to determine a session type, or the user desires to select an already known particular session type. FIG. 10 illustrates how the survey option asks, through the user interface, whether the user desires to choose their own audio/light content track. An affirmative answer results in a table of contents (list of tracks) being presented while a negative presents questions associated with a track identification process focused upon selecting among typical goals (stress reduction, ability to concentrate, energy level, mood improvement and sense of well-being), which will then synchronize the user's biofeedback data with their session selection to curate a track/session which correlates to their current needs and desires for the relaxation session.

The implemented user interface may be experienced as text questions and tactile (“button” pushing) answers through a flat panel touch screen (as in item 7 of FIG. 1), a similar interaction involving the user's personal smart phone appliance, synthesized or recorded voice commands and automated voice recognition of verbal responses, or any similar functional combination of such processes. A specific prerecorded audio track of relaxation content is selected based upon the user's answers to queries about goals for the immediate session. Simple example goals may include: help focus and increase mental performance, or reduce stress and relax, or increase well-being, and such other topics as will occur to those practitioners active in the field. An additional query may determine if the user is new to the apparatus or a return visitor. In the case of a new user, the track may play for about seven minutes and then halt, whereupon the user interface queries whether the track should be continued (typically with a decision about whether for 20 or 30 total minutes, for example) or an alternate track started. In the case of a returning user, the user interface ascertains whether the user desires a short session duration or a long session duration (e.g. 20 minutes versus 30 minutes, for example). Practitioners in the field of assisting relaxation generally agree an uninterrupted audio track duration of at least twelve minutes is needed for a typical user to have a fully beneficial experience. The duration of a session may depend upon details of the specific audio track being played (to avoid abrupt truncation of a musical passage, for example) and may also differ among tracks. The precise duration of short and long sessions may be adjusted (e.g. increased substantially beyond 30 minutes) according to user comments, commercial considerations, severity of the user stress being relieved, etc. The audio content may be delivered to the user through stereophonic headphones (either noise cancelling or not) as illustrated in FIG. 6. With sufficient sound adsorbing insulation in the dome, audio content may also be delivered through suitable loudspeakers embedded within the enclosure (chair) adjacent opposite sides of the user's head, although binaural beats generally require the bilateral acoustic isolation provided by headphone.

In another embodiment prerecorded audio content is played through the audio transducers in a determined sequence responsive to actions of the user upon the user interface. The prerecorded audio content may additionally contain binaural beats. The audio relaxation content may include one or more common elements such as spoken instructions or evocative suggestions, music, mantra meditations, guided relaxation content, various tones or gong sounds, ocean noises, and similar material. For added impact, the audio content may include monaural beats, isochromic beats, or binaural beats information, which are intended to affect the brainwaves of the user. Isochromic beat effect is achieved by the provision of a single tone (monaural beat) delivered with rapid interruption by silence, resulting in a staccato sound. The binaural beats effect is a form of pseudo-audible information produced within the listener, and may be achieved by techniques such as providing similar, but not identical, tones to each ear (the tone difference resulting in the desired beat frequency), or inverting the phase of stereophonic music channels at similar but not identical rates, or other known methods. The binaural beats may be chosen to match the relative frequency of various brainwaves the session intends to impact. Regarding association of audible information, or pseudo-audible information with brainwaves, practitioners in the art divide brainwaves into several categories, as described in Table. 1, although the frequency bands are not strict definitions and there is variability among authors and among users.

TABLE 1

| brainwave | frequency range | associated nominal |
|---|---|---|
| conventional name | (Hz) | consciousness condition |
| Delta | 0.1-3 | deep sleep |
| Theta | 4-7 | deep relaxation & |
| | | increased learning |
| Alpha | 8-15 or 8-13 | alert relaxation |
| Beta | 16-31 or 13-26 | alert concentration & |
| | | problem solving |
| Gamma | 32-100 | focused attention |

Audio relaxation tracks chosen for specifically different goals may nonetheless involve similar brainwave types based upon the nature of cognitive processes. The dome structure, or dome lid as case may be, is intended to be a non-resonant passive element which allows the audio relaxation content to directly impact the user experience without modification by the apparatus structural characteristics. The apparatus includes a function of isolating the user from an adjacent, potentially hectic, human social environment while enabling delivery of the audio content to the user within a comfortable individual microenvironment.

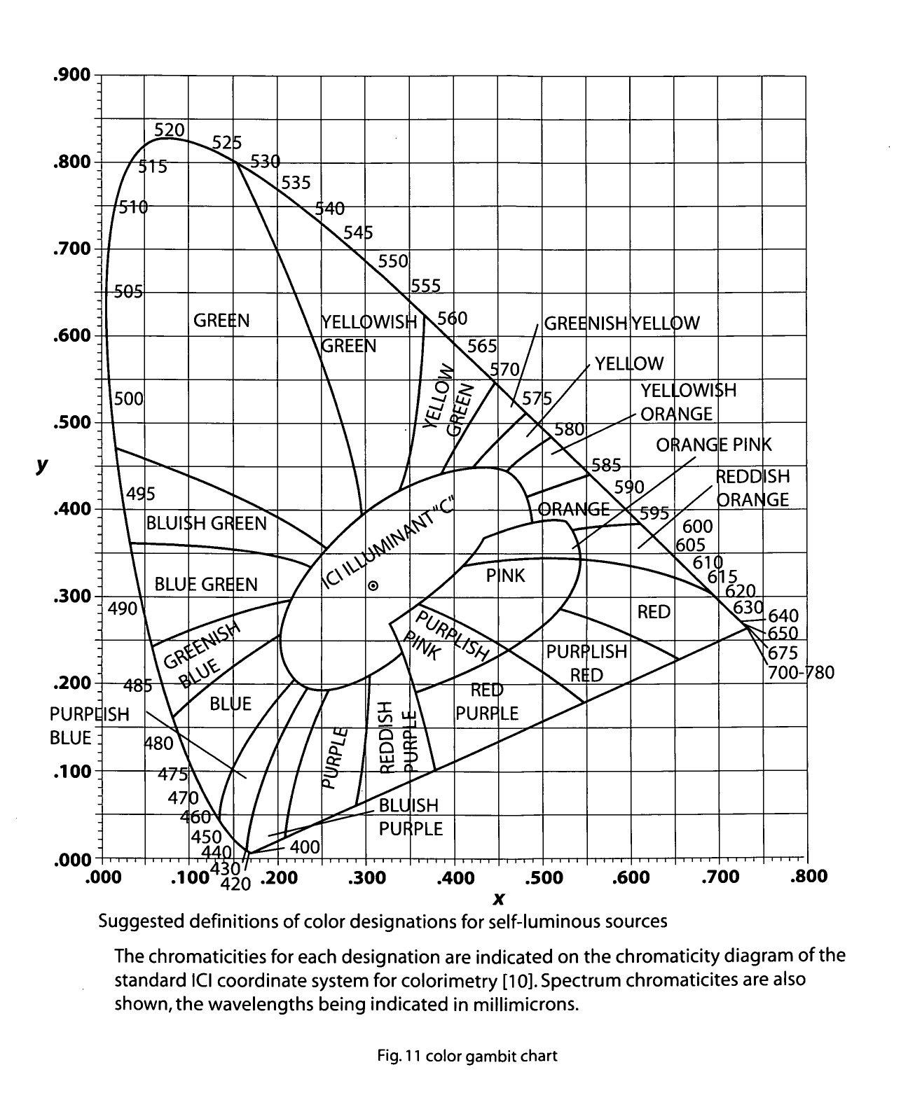

Light therapy (or chromotherapy) may be provided by a plurality of active emitters of visible wavelength light, typically located along the interior of the dome environment, providing chosen colors picked with consideration of the photometric response of the human eye. Previous users have reported the perceived colors of dark blue, light blue, green, and fuchsia (purplish red), to be preferred when used in conjunction with relaxation audio content tracks containing binaural beats. The field of color science is old, while also being both deep and broad, with developments continuing as new sources of illumination evolve. The November 1943 work of Kenneth L. Kelly, “Color Designations for Lights”, Research Paper RP1565 from the U.S. Department of Commerce National Bureau of Standards is a reasonable framework for understanding the preceding color designations (see FIG. 11). A typical compliment of active emitters of visible light may include light emitting diodes (LEDs) chosen to provide nominally red, green, and blue colors. Commercially available LEDs (available from Cree Inc. of Durham, N.C., for example) may have spectral outputs centered at approximately 458 nm (nanometers) wavelength for blue, 528 nm for green, and 625 nm for red. Note that according to FIG. 9 it is necessary to have both blue emitters and red emitters active simultaneously to achieve the perception of fuchsia (purplish red). The perceived brightness of each emitter depends upon the wavelength of that emitter due to variations in the sensitivity of the human eye, and the emitters have wavelength dependent power conversion efficiency. One common LED control scheme is to excite a LED with a constant current. Classic switchable (on/off) circuits for such a function are well known. The resulting perceived light intensity may be changed by tune domain modulation of the nominally constant LED current using a repetitive on/off command sent at a repetition rate sufficiently high to be unperceivable to the human eye (e.g. 100 Hz). The greater the fractional “on” period then the brighter is the perceived contribution of the particular visible light emitter. With typical LEDs a scenario for creating a fuchsia color might be a 50% duty cycle of nominal LED design operating current being sent to both the blue emitter and the red emitter. Apparatus designers may subsequently choose to adjust the lighting intensity by adjusting the duty cycles and tune the hue by making the blue and red duty cycles different.

A stored program electronic computing device with appropriate analog and digital input-output hardware is contemplated for typical implementations of an apparatus control device. The output of the control device includes color information directing illumination dispersed through the dome interior which fills the user visual field, and also includes audio information which is provided through the stereophonic sound delivery elements (headphones being preferred in the event the audio content includes binaural beat information). The stored program directing apparatus control device functions may include menu constructs for the user interface or menus may be obtained from a database by action of the stored program. In any case, the user interface menus may include convenient colloquial names used as references to specific combinations of light therapy color and binaural beat audio content as shown in Table. 2.

TABLE 2

| | | | brainwave type |
|---|---|---|---|
| example | | | matched by |
| menu name | user relaxation goal | perceived color | binaural beat |
| Recharge | invigorating track for | light blue | beta, gamma, |
| | focus & promoting | | theta |
| | creativity | | |
| Relax | replenishing track to | light blue | alpha |
| | calm mind & relieve | | |
| | stress | | |
| Focus | increase energy & | dark blue | beta, gamma, |
| | mental acuity | | theta |
| Overcome | assist struggle against | green | theta |
| | addictions, increase | | |
| | energy & metabolism | | |
| Heal | release Human | green | delta |
| | Growth Hormone, | | |
| | boost immune system | | |
| | & wellbeing | | |
| Love | improve clarity, | fuchsia - light purple | theta |
| | increase charisma | | |
| Manifest | cultivate | fuchsia - light purple | theta |
| | connectedness & | | |
| | attraction | | |
| Succeed | inspire, improve | dark blue | theta |
| | mood & self esteem | | |

Consideration of Table. 2 reveals it is feasible to guide a relaxation apparatus user toward slightly different personal goals, using very similar audio and visual stimuli, by changing the consciously heard audio track content. For example, both the “Love” and “Manifest” menu choices result in the control device presenting fuchsia color light (a combination of red and blue as previously discussed) and audio content tracks having binaural beats intended to entrain the user toward theta brain waves, but the music, suggestive voice-over, and other nuances of the audio content will be specific to particular track (“Love” versus “Manifest”). Other menu options and corresponding combinations of light and sound stimuli may be arranged, as will occur to those practitioners active in the field, using constituent definitions shown in Table. 3.

TABLE 3
Green - Affects blood pressure and conditions of the heart. Stimulates
rejuvenation and addresses hormonal imbalances. Cleans and purifies
from germs, bacteria, and rotting material.
Harmonizes digestion, stomach, liver, gallbladder, and kidney health.
Increases immunity & stimulates inner peace, while strengthening the
nervous system.
Light Blue - Cooling and calming. Helps to relieve headaches, high blood
pressure, and stress, while calming intense emotions such as anger.
Can help with ailments associated with speech and communication.
Dark Blue - Relaxes sensations of stress while improving intuition and
sensitivity. A color for non-triviality, renewal, innovation, and alternative
ways of living.
Fuchsia - The color of transformation, spiritual insights, and renewal.
Can help to slow down an over active heart, and stimulate the spleen and
white blood cells for immunity.
Alpha - for heightened creativity and deep relaxation; reduce cortisol,
boost immune system, increase DHEA (mood enhancing), and increase
melatonin to help with insomnia
Beta - for focus and concentration; shown to reduce symptoms of ADD &
is the state for hyper-focus and cognition
Theta - for meditation; insight and gateway to super learning & long-term
memory, dream recall
Delta - for deep sleep and healing release of HGH (human growth
hormone), deeply restorative rest & rejuvenation, accelerates healing
Gamma - to increase cognition and improve memory, natural
antidepressant, relieves symptoms of ADD & ADHD

The system may provide an opportunity for a user to modify their experience based on information collected at various stages of use. Users may make survey choices which modify selection opportunity, or audio content track selections prior to initiation of a session which guide use of the invention during that session. A scheduling method may also permit initiation without choices upon entering the apparatus. Users may also employ a data-collection strategy which connects their experience to a longitudinal system such as via their smartphone, or other personal data device, or via a web-based Internet portal, allowing the user to input data requesting a session based on a variety of needs they are subjectively experiencing: tired, stressed, under the weather/sick, needing to focus, desiring a mental boost/inspiration, as well as other targeted sessions. This user input is analyzed by the control device to determine which session selection will best suit the needs of the user for the current session. The control device may also recognize the user's identity based upon previous profile data collected, and enable said user to reference their progress from prior sessions in comparison to their current session. Information is provided from the user to the control device in the form of subjective and objective data from a plurality of sources including but not limited to survey response, personal selection or control guidance information (i.e. audio content track choice, audio volume, start/stop commands, etc.), as well as demographic and other information as provided by the user according to an apparatus use agreement. Since the apparatus is intended to generally provide users can automatically guide relaxation experience without the presence of an attendant clinician, there is usefulness in the user interface and control device providing an electronic calendar function for session scheduling among multiple users. Apparatus control device connection to databases, as described further below, enables implementation of scheduling algorithms whereby a user may reserve access to the apparatus at future dates and times. Apparatus control device connection to the Internet further enables access scheduling through transaction processes using World-Wide-Web user interface techniques or mobile communication device (e.g. cellular telephone or tablet computer) application programs (“apps”) as is known to practitioners skilled in those arts.

A biofeedback or neurofeedback and control device system may be provided with the microenvironment described above as part of the invention. The apparatus structure will reduce or eliminate ancillary and unintentional, or merely present, audible and visual stimuli not desired by a user. Biofeedback or neurofeedback information may be collected which 1) demonstrates physiological markers, 2) demonstrates neurophysiological markers, 3) allows identification of performance ranges of said markers, 4) permits the association of these ranges with peak versus poor performance comparison according to demographic averages or medical literature controls for said markers or surrogate markers, 5) assists in the modification of audio and visual output data, 6) facilitates the user's efforts to modify thinking, or relaxation, or other behaviors (e.g. breathing, movement) towards an ideal state as preferred by the user. When the user has attempted to reach, or reached, a nominally sensory-reduced state, the control device may modify audible and visual information provided by the apparatus as already described above according to information collected from the user within the apparatus. This relaxation apparatus may thus include continuously-variable instantaneous feedback.

Information collected during an active session from the user may include pulse rate, respiration rate, blood oxygen saturation, body temperature (especially extremity temperature or variability), body and extremity movement as a surrogate marker for psychomotor agitation, heart-rate variability (HRV) and power spectrum density (PSD) or other surrogate markers from primary HRV data, galvanic-skin response (GSR), electromyography (EMG), electrocardiography (ECG), electroencephalography (EEG) including frequency band activity (delta, theta, alpha, beta, gamma) and sensory motor rhythm (SMR), homoencephalography or hemoencephalography (HEG), or other physiological or neurological actual or surrogate data. The user would place sensors upon their body or head, or expose themselves within the apparatus to similar contained sensors at appropriate locations to collect this information, and participate in an audio-visual session within the apparatus. The apparatus could also make use of secondary data obtained from primary sensor data, for example power spectrum density (PSD), independent component analysis (ICA) or group ICA, current source density (CSD) of EEG scalp potentials and subsequent analysis of spectral power, Laplacian or Fourier or other transformations, and other methods of analysis known to those skilled in the art of digital signal processing or clinical neurophysiology. Heart rate variability (HRV) is one example of secondary data which may be obtained, via mathematical analysis, from primary sensor data such as simple pulse detection with a clip-on finger sensor. These data may be used for simultaneous output variability affecting the primary session of the apparatus, or it may be used to generate info-graphic information to be displayed to the user in real time, or for longitudinal studies by the user or on the user's behalf via the apparatus, or via devices connected through the control device as selectively allowed by the user.

An aspect of the controller may include one or more user states according to collected information. For example, pulse rate and respiration rate could be collected on initiation of a session and at close of the session for pre/post session comparison of the user's so-defined “cardiopulmonary” state. For users who were comfortable with continuous monitoring, heart and respiratory trends during a session could be monitored for comparison to audio content track information within the session that may correspond to brainwave states associated with relaxation. Thus a “relaxation” state, for example, could be generated for the user according to any portion or all information collected by the control device during a session as described above. After the session, i.e. for post-hoc analysis, the user could evaluate their “performance” within the session as part of an ongoing relaxation strategy. This information may also be compared or contrasted against existing averages, or existing ideals, for a user based on demographic data including height, weight, age, sex, degree of athletic activity, etc., or based on a-priori biofeedback or neurofeedback information collected by the control device itself across sessions, providing further feedback to the user. Some information is separated from specific items potentially unique to the user (de-identified or anonymized) for optionally generating population data by users, or the user could choose to maintain a unique identification comparing their own information against user averages across sessions or within sessions. The user could conceivably choose an “offline” or an “inline” method for making use of this data, where an offline method would collect information but only for display to the user after the session was complete, and an inline method would make instantaneous use of the data during the session. A user may decide to pursue a relaxation method which brings their relaxation state towards the average relaxation state achieved by other people during the same session. In this simple example, relaxation state would be defined as a number value influenced by subset categories of pulse rate, respiration rate, skin temperature, and galvanic skin response. Subset boundaries based on physical human limitations for each physiological data are used to generate subset numbers contributing towards a defined relaxation state. For example, exists particular user's natural variation in pulse rate may be between 65 and 85 beats per minute (bpm), which does not itself represent degree or absence of relaxation. However, the control device could for example define 100 bpm (the medical definition of tachycardia) as a state demonstrative of a lack of relaxation, and ascribe a poor “relaxation” value for cardiac subset influence which approaches this number (e.g. greater than 90 bpm). Likewise, the presence of a relaxation process or state could be defined by a pulse rate below a certain absolute limit (e.g. less than 60 bpm, which is the medical definition of bradycardia), or it could be defined according to a percent reduction in pulse rate compared to that user's baseline, or compared to average user reduction during the same period. As such, a user could still be defined as “relaxed” if their degree of pulse rate decrease exceeded the percentage expected based on simply lying still, or in this case if it coincided with user averages, even if it did not reduce below an absolute number. Similar subset value boundary conditions can be ascribed for each biofeedback or neurofeedback input collected. More complicated examples provided by the apparatus are inferred by the presence of additional sensors and provide the opportunity for more robust experiences using the apparatus. For example, EEG background frequency data could be collected, or could be used by the device without collection (given the potentially large volumes of data involved) to demonstrate brainwave entrainment (BWE) of baseline activity with binaural beat patterns provided by the unit, with or without other audio or visual such as photic or other stimulation. In this way a session could include user brainwave state to modify session activity towards a desired user brainwave state as selected by the user.

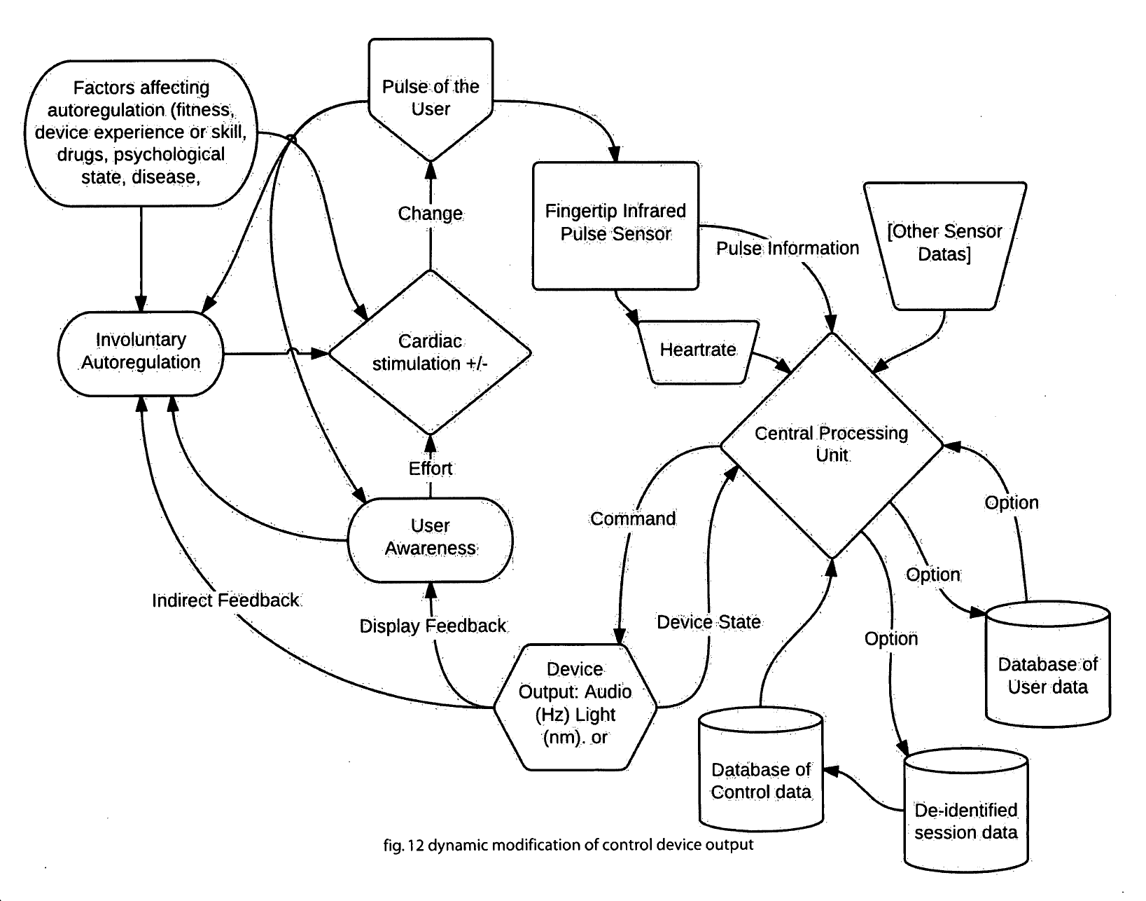

Given a user's a-priori choice towards a relaxation state based on the average state of all users, the control device would chart the individual and combined biofeedback or neurofeedback information and modify the audio and visual stimuli presented to the user (thus modifying the “user experience”) whenever there was deviation from nominal (i.e. “relaxation” state of the average user). As such, the user would train the control device to make changes in the session automatic control program according to predetermined intentions of the user engaged in the specific self-directed relaxation session. Examples of changes made by the control device could include within-session modification of audible information. For example, if absolute relaxation state were observed by the control device to decrease (i.e. pulse rate went up, respiration rate went up, elements of heart-rate variability increased, galvanic skin response increased, peripheral skin temperature decreased), or if user relaxation was reduced compared to the average user condition at the similar moment during the particular session audio content, or if increased EEG beta activity associated with increased attention in numerous cortical areas were detected or other EEG aggregate information were calculated to demonstrate diffuse global cortical activity suggesting a lack of relaxation then the control device could make changes that were predicted to have an improved outcome on relaxation. For example, the control device may employ a binaural beat pattern more associated with alpha brainwave states, or it could reduce light intensity or change perceived color, or it could attempt to entrain the brain towards the existing relaxation program with sub-routines meant to increase attention, such as the overlay of a brief vocal reminder to slow breathing, or an isochromic tone associated with brainwave entrainment, or high frequency intermittent photic stimulation, or other methods. Transactions with databases may be conducted through a central processing unit which in turn communicates with the control device that manages the microenvironment presented to the relaxation apparatus user. The central processing unit, and physical storage media for the databases, may be located far away from the relaxation apparatus and communicate through a suitable Internet connection (so-called “Cloud” computing situation). Alternatively, the central processing unit and control device functions might be effectively merged and be performed by computing hardware located adjacent or within the relaxation apparatus. A flowchart illustrating the above discussed elements of sensor biofeedback including continuous user data, previously collected data from the user, data from historical or other user data controls, central processing unit control, and control device output modification is included as FIG. 12 (flow chart illustrating dynamic modification of control device outputs to the user microenvironment based upon real-time physiological biofeedback data combined with historical data of both the specific user and general populations).

One of the biofeedback and control device system is the ability to predict the needs of the user based on the presently detected state of the user. This functionality may include auto-selection for the session based on user state, or change in the color of the light emission, or be used for ongoing real-time monitoring within a selected session. The predictions may be inferred via baseline measurement using apparatus sensors, possibly while considering individual user profile information, with or without subjective survey input described above. An example is provided in the form of stress as represented via biofeedback surrogate markers, with the presumption that relaxation is a form of reducing physical stress, and with the further presumption that said relaxation may be measured as represented by changes in those same biofeedback markers. Algorithms in the control device (or alternatively the central processing unit) define metrics for stress, degree of relaxation, and other measures, based in part on the results of practical research with the relaxation apparatus while considering physical limits of sensors, normal range of variation among the general population, and the ability of the control device to do simultaneous calculations (or ascribe assumptions when signal artifacts impede calculation) and other complications known to those skilled in the technical craft of biofeedback art.

For example, user state of stress would exist as a variable, [STRESS], which is a number arrived at via calculation of subcomponent variables, with each of those subcomponent variables having definitions assigned to them which, when exceeded or reached, categorize that subcomponent as a contributor to the stress variable or, conversely, when reduced or diminished, detract negatively from the assigned value of the stress variable. For example, in an otherwise healthy adult user between 12 to 55 years of age, heart-rate above 100 beats per minute (bpm) is defined as tachycardia, and so described indicative of the presence of stress, while less than 60 bpm would define bradycardia, and the presence of a relaxed state. Respiratory rate above 20 breaths per minute would indicate tachypnea, and so described would contribute to the calculation of stress, while less than 12 breaths per minute would define a state of bradypnea. A positive correlation with the presence of an ongoing relaxation process could be represented in this example by a 20% reduction in the heart-rate of the user based on the difference of session baseline recorded heart-rate over that individual's nominal resting value as described by previous recording sessions, or based on the difference of baseline heart-rate above user control averages for weight and age, or based on the heart-rate over an absolute value such as 60 bpm. The actual calculation used will vary per user, per recording, or per session output variable. For example, when defining physiological stress as an output variable, normal limits for heart-rate would be defined as above (60-100 bpm); however, when used in a representation of emotional state, heart-rate may not be as important in absolute number as the beat-to-beat variability of the heart-rate, and the resulting mathematical transform of that rate as known to those in the biofeedback art as heart-rate variability (HRV) and power spectral density (PSD). Likewise, data measures representing brainwaves along the frequency profiles represented elsewhere within this document (alpha, beta, etc.) may be correlated with those binaural beat patterns for the purposes of entrainment along with audiovisual stimulation, while using other biofeedback metrics.

Using examples of input variables from the apparatus' sensory matrix such as but not limited to those described above and elsewhere within this document for heart-rate, HRV/PSD, background brainwave frequency, and subjective user selection or survey response, and using output variables such as binaural beat pattern session selection and device color wavelength for chromotherapy, the relaxation apparatus will modify audiovisual output based on device inputs. For interpretation of real-time data, and due to limitations in sensor collection capability due to signal noise or applicability within the general population for non-normal variables, or other limitations known by those in the biofeedback and medical arts, it may be necessary to transform primary sensor information into secondary information for calculation, and to relate those secondary and primary information variables to each other in the form of a calculation for the purposes of producing representative surrogate markers for the variable of interest, for example mood, emotional state, brainwave state, stress, somnolence, intention, ability to concentrate (attention), and et cetera. Consider the below summarized example:

If wavelength of light were the output variable [OUTPUT=LIGHT(nm)], and input variable was [STRESS], with combined input variables of heart-rate variability [INPUT1=HRV(PSA)], beat frequency [INPUT2=BEAT(Hz)], respiratory rate [INPUT3=RR(bpm)], and subjective report of stress at entry based on a scale of 1-10 [INPUT4=USERSTRESS], then we could report:

### OUTPUT=INPUT

OUTPUT=(function of)(INPUT)
OUTPUT=f(INPUT)=[f(INPUT1)+f(INPUT2)+f(INPUT3)+f(INPUT4)]
(change in nm) LIGHT=f(STRESS), or f(HRV)+f(BEAT)+f(RR)+f(USERSTRESS)

The apparatus control device would cause a change to the perceived wavelength (nm) of visible light emitted into the dome environment as disturbed by an amount representing the recorded stress in that user, as defined by the recorded components of physical stress used in our example (RR, HRV), and inclusive of sound being provided to the user, as well as the subjective stress reported by that user. Therefore, for a degree of change in stress level (or, conversely, amount of observed relaxation per those biofeedback measures), and related to the brainwave state expected based on the binaural beat frequency being provided or as potentially reported by EEG sensors, and as inclusive of the subjective information for stress provided by the user during a baseline or online survey for mood, there would be an associated change in the color output of the dome. In this way, a person who reported a higher degree of stress, or who had biophysical markers which demonstrated a higher degree of stress, would be shown a color wavelength further within a presumed treatment spectrum for a higher stress level. Therefore, a user may have real-time feedback metrics which represent calculated values for relaxation, which when recorded are shown to appear co-incidentally with binaural beat patterns also representative of relaxed mental states, and when these values are demonstrated by the user as correlating temporally the apparatus would define the user as having reached a relaxed state, and change the color of the dome to appropriately represent this change in state, providing visual feedback to the user, while also providing a data metric marking (in the ongoing recording) that change in state to permit the user to identify physical biofeedback traits present at this now “marked” moment in time. A worked example is described below:

An example: user enters the apparatus and requests for a guided session, placing biofeedback or neurofeedback sensors as appropriate, and choosing an audio content track that plays audible content expected to be associated with theta and alpha brainwave activity within the user. During the introduction of the session the control device makes a 30 second recording of data from the biofeedback sensors to establish a baseline relaxation state calculation. This example user has a pulse rate of 105 bpm, a respiration rate of 24/min, a heart-rate variability with a high degree of fluctuation based on existing limits within the control device, and a high galvanic-skin conductance which here is interpreted to represent increased adrenaline sympathetic activity, all of which are used to generate a baseline value for the example user indicating an increased degree of underlying physical stress compared to historical controls. As the session begins, the example user exhibits a degree of relaxation expected given the change in physical state including the reduced work of lying still, controlled breathing, and enjoying the audio track. The user's level of relaxation (i.e. reduction in stress) is measured using continuous biofeedback data to generate ongoing surrogate markers for physical stress that may be compared to historical controls, or compared to similar data collected from average device users. Our user improves consistently, and within-range of average users. The example user is now 5-minutes into a session, and binaural beats are being provided at 5 hz (˜theta) and 12 hz (˜alpha) frequency oscillations consistent with session goals chosen at initiation. However, the example user begins to become distracted, and engages in anxious ruminating cognitive behaviors while mentally reliving an argument from the workplace at a meeting earlier in the day. Biofeedback measures via the user sensors are observed to have deviated from projected nominal, and specific neurologic measures report higher power in slow EEG bands (i.e. theta) with lower power in high EEG bands (i.e. Beta) suggestive of a distracted state as is commonly seen with neural correlates of attention-deficit. The control device recognizes this state as inconsistent with session goals and employs a session modification subroutine. This subroutine will attempt to redirect user attention towards the session experience, and need not specifically be revealed to the user as having been initiated (e.g. in this example the user remains naive that an observation of increased stress has been made, although specific feedback to the user of ongoing stress level may also be a component of the device). The audio channel volume on the already playing binaural beat is reduced slightly and volume of a new track specific to the subroutine is increased to levels that have been shown to be cognitively audible, without overt distraction or inconsistency within the original track. This particular subroutine audio track employs a binaural beat frequency which is associated with attention (beta, ˜30 Hz), and then replaces it with a frequency associated with non-REM sleep and lack of body awareness (delta, <4 Hz). A light photic stimulation is briefly engaged to increase user attention, while modification of the dome backlighting hue is made towards a softer (less saturated) shade of the current color. The user experience is such that the example user is distracted from their anxious ruminating behavior and attention is re-engaged in the ongoing session, permitting them to continue the relaxation task. Biofeedback and neurofeedback metrics collected during this period of session modification will later demonstrate not only that the example user has returned to a nominal state, but also indicates that the general relaxation value for this example user trended downwards on average over the course of the 1-minute session modification subroutine, allowing the user to have experienced a “guided” session. Notably, in this example case study, domains of heart-rate variability interpreted as stressful, and skin galvanic response as isolated values paradoxically increased to the excess of a degree explained by normal variation or recording artifact as compared to average users or to historical controls during brief periods of ˜30 Hz beta stimulation, suggesting the possibility that this example user is more susceptible to that beat frequency than an average user. This within-user data could be accumulated over time as the user develops an ongoing relationship with the relaxation apparatus, allowing subroutines to be individually catered towards expected response state given previous observations, rather than compared to historical controls. Based on the desired presence or absence of a sensitized attention response according to evolving user goals, relaxation values for this user could modify according to evidence-based responses to previous recorded stimuli, and in a future session the attention stimulation subroutine could modify with a reduced intensity, volume, or duration according to the expectation that this user has shown a previous paradoxical response. The within-session data for the user within this example will also permit modification of future sessions. For example, accumulation of aggregate data across multiple sessions might reveal that although the example user had early experiences averaging 6-10 stressful deviations from nominal relaxation during a 25 minute session, recent sessions were completed without the initiation of any re-entrainment subroutines. The example user is also able to identify that resting heart-rate at baseline evaluation during the beginning of a session has reduced by 22 bpm, insinuating an average degree of physical relaxation which precedes device initiation according to the surrogate marker of heart-rate, suggesting anticipatory relaxation with device usage over time. Further, for this user during a future retrospective and by combining survey data, it is observed that a state of nominal relaxation based on all recorded values was originally achieved during the primary session only after 18-minutes, but with continued use has been achieved at 3-minutes, which is 1-minute faster than the average rate when compared to average users of similar age, sex, weight, coffee consumption, and number of sessions.

1. A method of assisting an individual to achieve a relaxed mental state whereby the individual is presented a session comprising a predetermined combination of simultaneous auditory and visual stimuli while being inside a self-contained private space.

2. The method of claim 1, wherein the session includes an uninterrupted duration of at least twelve minutes.

3. The method of claim 2, wherein visual stimulus is of a substantially deep-blue color and the auditory stimulus includes binaural beats approximating the frequencies of beta brain waves and gamma brain waves and theta brainwaves.

4. The method of claim 2, wherein visual stimulus is of a substantially light-blue color and the auditory stimulus includes binaural beats approximating the frequencies of beta brain waves and gamma brain waves and theta brain waves.

5. The method of claim 2, wherein visual stimulus is of a substantially green color and the auditory stimulus includes binaural beats approximating the frequencies of delta brain waves.

6. The method of claim 3, wherein visual stimulus is of a substantially green color and the auditory stimulus includes binaural beats approximating the frequencies of theta brain waves.

7. The method of claim 2, wherein visual stimulus is of a substantially light-blue color and the auditory stimulus includes binaural beats approximating the frequencies of alpha brain waves.

8. The method of claim 2, wherein visual stimulus is of a substantially fuchsia color and the auditory stimulus includes binaural beats approximating the frequencies of theta brain waves.

9. The method of claim 2, wherein visual stimulus is of a substantially deep-blue color and the auditory stimulus includes binaural beats approximating the frequencies of theta brainwaves.

10. A stand-alone relaxation unit, comprising:
an binaural beat audio generator;
an light therapy device;
a chair;
a housing integrated with the chair, the housing defining a partially-enclosed personal space.

11. The relaxation unit of claim 10, further comprising a controller adapted to select an audio content based on a biofeedback of a user, the controller further adapted to cause the audio generator to emit the audio content.

12. The relaxation unit of claim 11, wherein the controller is further adapted to select a light therapy content based on a biofeedback of the user, and to cause the light therapy device to emit the audio content.

13. The relaxation unit of claim 12, wherein the light therapy device includes an LED coupled with a dome of the housing.

14. The relaxation unit of claim 13, further comprising a ventilator.

15. The relaxation unit of claim 11, wherein the biofeedback is a datum selected from the group consisting of a pulse datum, a rate of respiration datum, a blood pressure datum, and a neurosensory datum.

16. The relaxation unit of claim 12, wherein the controller is further adapted to cause the biofeedback to be recorded.

17. The relaxation unit of claim 13, further comprising a TRH4 disc coupled with the dome.

18. The relaxation unit of claim 16, wherein the controller is adapted to select an audio content based on a recorded biofeedback of the user of a prior session.

19. The relaxation unit of claim 18, wherein the controller is adapted to present a progress report based on the recorded biofeedback of the user of the prior session.
